(19) United States
(12) Patent Application Publication
(10) Pub. No.: US 2012/0209313 A1
(43) Pub. Date: Aug. 16, 2012

(54) Nasal Dilator With Means To Direct Resilient Properties

(76) Inventor: Joseph Vincent Ierulli, Portland, OR (US)

(21) Appl. No.: 13/437,929

(22) Filed: Apr. 3, 2012

Related U.S. Application Data
(63) Continuation of application No. 13/206,462, filed on Aug. 9, 2011.
Application No. 13/206,462 is a continuation of application No. 12/106,289, filed on Apr. 19, 2008, now Pat. No. 8,062,329.
(60) Provisional application No. 60/913,271, filed on Apr. 21, 2007.

(51) Int. Cl. A61F 5/08

(52) U.S. Cl. 606/204.45

(57) ABSTRACT

A nasal dilator comprises a laminate of vertical layers that form a unitary, or single body, truss having horizontal regions adapted to engage outer wall tissues of first and second nasal passages and to traverse the bridge of a nose therebetween. When in use the dilator acts to stabilize and/or expand the nasal outer wall tissues and prevent said tissues from drawing inward during breathing. The dilator includes multiple parallel resilient members or a resilient member having a plurality of component spring fingers extending from a common center. The dilator may further include material separations, or discontinuity of shape of material, formed in at least one region of the truss and extending through at least one layer of the dilator.

### RELATED APPLICATIONS

The present application is a Continuation In Part of U.S. Non Provisional patent application Ser. No. 13/206,462, filed 9 Aug. 2011. Non Provisional patent application Ser. No. 13/206,462 is a Continuation of U.S. Non Provisional patent application Ser. No. 12/106,289 filed 19 Apr. 2008. Non Provisional patent application Ser. No. 12/106,289 claims priority benefit from Provisional Patent Application No. 60/913,271 filed 21 Apr. 2007.

### FIELD OF THE INVENTION

The present invention relates to apparatus and methods of dilating external tissue. As disclosed and taught in the preferred embodiments, the tissue dilator devices are particularly suitable for use as external nasal dilators for supporting, stabilizing, and dilating nasal tissues adjacent and overlying nasal airway passages of the human nose, including the nasal valve and/or the vestibule areas thereof.

### BACKGROUND OF THE INVENTION

A portion of the human population has some malformation of the nasal passages which interferes with breathing, including deviated septa and swelling due to allergic reactions. A portion of the interior nasal passage wall may draw in during inhalation to substantially block the flow of air through the nasal passage. Blockage of the nasal passages as a result of malformation, symptoms of the common cold or seasonal allergies are particularly uncomfortable at night, and can lead to sleep disturbances, irregularities and general discomfort.

Spring-based devices for dilating outer wall tissues of the human nose are disclosed in U.S. Pat. Nos. 6,453,901; D379,513; D429,332; D430,295; D432,652; D434,146; D437,64; and 8,062,329; the entire disclosures of which are incorporated herein by reference. The commercial success of at least one of these inventions, together with that of other modern external nasal dilators, collectively and commonly referred to as nasal strips, has led to the creation and establishment of a nasal dilator product category in the present consumer retail marketplace. Commercial success of prior art nasal dilator devices disclosed before 1990, in particular that of U.S. Pat. No. 1,292,083 (circa 1919), is presumed to be commensurate with the nature of consumer product retail environments at the time of those inventions.

Throughout the history of those medical devices which engage external bodily tissue (i.e., tissue dilators, nasal splints, ostomy devices, surgical drapes, etc.), a long-standing practice in the construction and use thereof has been to interpose a buffer material between the device and the user's skin to facilitate engagement of the device to the skin and to aid user comfort. Said material, such as a spunlaced polyester nonwoven fabric, typically has properties which permit limited, primarily plastic and somewhat elastic deformation within the thickness thereof. These properties can spread out peeling, separating or delaminating forces such as may be caused by gravity acting on the weight of the device; the device's own spring biasing force or rigidity (such as that of a tissue dilator or nasal splint); biasing force that may be present in bodily tissue engaged by the device; surface configuration differences between the device and the skin of the device wearer; displacement of the device relative to the skin or external tissue as a result of shear, tensile, cleavage and/or peel forces imparted thereat via wearer movement (e.g., facial gestures) and/or contact with an object (e.g., clothing, pillow, bedding, etc.); and so on, that may cause partial or premature detachment of the device from the wearer. By spreading out these delaminating forces, said interface material acts as a buffering agent to prevent the transfer of said forces to its adhesive substance, if any, and thereby to the skin. Preventing the transfer of focused delaminating forces substantially eliminates any itching sensation (caused by the separation of the adhesive substance or device from the skin) that a wearer may experience if these delaminating forces were otherwise imparted directly to the skin.

There has been a continuing need in the art to develop nasal dilators which address and improve upon the dynamics and design parameters associated with limited skin surface area adjacent the nasal passages, adhesive attachment, delaminating spring biasing forces, device comfort, and durational longevity.

Tissues associated with and adjacent the nasal passages have limited skin surface areas to which dilation may be applied. Said surfaces extend upward from the nostril opening to the cartilage just above the nasal valve, and extend outward from the bridge of the nose to each approximate line where the sides of the nose meet each cheek.

Nasal dilators are, of necessity, releasably secured to said skin surfaces by use of pressure sensitive adhesives. Skin surfaces transmit moisture vapor to the surrounding atmosphere. Said adhesives break down in the presence of skin oils, moisture and the transmission of moisture vapor, often within hours.

External nasal dilator devices of the present modern era feature a flat, substantially rectangular or slightly arcuate resilient member made of plastic. When engaged to a nose, the resilient member exerts a spring biasing force which tends to substantially return or restore the device to an original, generally planar, state thus dilating the local tissue. Said spring biasing force creates primarily peel and some tensile forces generated at the end regions of the device where engaged to the nose of a wearer. Said forces work to delaminate the end regions of the dilator device from skin surfaces so engaged.

Constructing a device with less than 10 grams of spring biasing force in order to mitigate delaminating peel forces may not provide suitable stabilization to, or dilation of, nasal outer wall tissues. Over-engineering the dilator by using a more aggressive adhesive, a greater amount of adhesive, or greater adhesive surface area in order to withstand greater spring biasing force increases the likelihood of user discomfort during use and damage to the tissue upon removal of the device. Additionally, a dilating spring biasing force of 40 grams or more could, in and of itself, be uncomfortable for most users.

Presently known spring-based nasal dilator devices which are suitable or adaptable for mass commercialization include devices disclosed in U.S. Pat. Nos. D379,513; 5,533,503; 5,546,929; RE35408; 6,453,901; 7,114,495; and Spanish Utility Model 289-561 for Orthopaedic Adhesive. These devices provide sufficient dilation of nasal passage outer wall tissues and thus provide the claimed benefit to the vast majority of users. In addition, the '503 and '901 disclosures teach means for shifting, transforming and redistributing delaminating peel and tensile forces into primarily shear forces. Said shifting or transforming is desirable since the pressure sensitive adhesive disposed on nasal dilator devices for engaging skin surfaces adjacent the nasal passages withstand shear forces generally better, longer and more reliably than peel forces.

The '901 disclosure teaches a simple end region structure in FIGS. 10-11 that includes relief cuts placed adjacent each terminal end of a single resilient spring band, extending around its terminal ends and slightly along the upper and lower longitudinal edges thereof, corresponding to the general outline of the terminal ends of the resilient band without contact thereto. When in use on the nose of a wearer, this structure shifts peel and tensile delaminating forces into primarily shear forces which are imparted to the material extending between said relief cuts and the lateral end edges of the device.

The '901 patent also discloses a nasal dilator in FIGS. 16-18 that features resilient spring fingers configured so as to provide dilating force to skin surfaces overlaying both the nasal vestibule and nasal valve. However, the fabrication process wastes more material than that which is devoted to the resilient member itself. U.S. Pat. No. 6,769,429 discloses independently flexible upper and lower finger elements diverging from one another. However, the fingers all curve beyond and terminate to the same side of the longitudinal centerline of the device. U.S. Pub. No. 2002/0000227 uses closely parallel spring finger components to exert tensing force in a direction parallel to the skin surface of the nose and the surface plane of the dilator. However, arriving at a suitable material and fabricating a resilient member that flexes in opposing directions—both parallel and perpendicular to it's long axis—is problematic. Accordingly, there remains a need in the art to provide nasal dilator devices having resilient member spring finger components that are both efficacious as well as economically and easily manufactured.

U.S. Pat. No. 5,611,333 discloses a dilator device that features various openings, slits, notches and cuts formed within the peripheral edges of a resilient member to selectively reduce spring biasing forces locally so that the resilient member may be used as a stand alone dilator device without the use of additional materials for maintaining the dilator device engaged to the nose of a wearer.

The present invention builds upon the prior art by providing means to direct the resilient properties of a nasal dilator whereby to overcome the aforementioned limitations specific to external dilation of the human nose.

### SUMMARY OF THE INVENTION

The present invention teaches, depicts, enables, illustrates, describes and claims new, useful and non-obvious apparatus and methods of providing dilation to external tissue. In particular, the present invention provides a wide variety of tissue dilators adapted to engage an exterior tissue region of a human nose to dilate the nasal passages thereof, including the vestibule and/or nasal valve areas. It is the principal objective of the present invention to provide nasal dilator devices which improve and build upon the prior art and address unmet needs in the art.

In the specification and claims herein, the term vertical refers to a direction parallel to the thickness of the dilator or truss. The term horizontal refers to a direction parallel to the length, or longitudinal extent, or long axis of the dilator or truss. The term lateral refers to the width or opposing end edges of the dilator or truss, or a direction perpendicular to the length, longitudinal extent, or long axis of the dilator or truss. The term longitudinal centerline refers to a line parallel to the longitudinal extent of the dilator or truss, bisecting the width of the dilator or truss midway between its upper and lower long edges. The term lateral centerline refers to a line perpendicular to the length, longitudinal extent, or long axis of the dilator or truss, bisecting the long axis, or upper and lower long edges, midway along the length thereof. The terms upper and lower refer to orientation between like objects, particularly with regard to plan views, as seen in relation to the top and bottom of the drawing sheet page.

The external nasal dilator of the present invention comprises a laminate of vertical layers. The laminated layers form a unitary, or single body, truss with each layer consisting of one or more members and/or components. The layers preferably include a base layer, resilient layer, and cover layer. Any single layer, or a combination of two or more layers may define the peripheral shape or edges of the dilator. The dilator is die cut from a continuous laminate of material layers, and dilator members or components may be die cut, in whole or part, from one or more continuous material layers before or during assembly of the continuous laminate. The truss features horizontal regions including first and second end regions adapted to engage outer wall tissues of first and second nasal passages, respectively, and an intermediate region adapted to traverse a portion of a nose located between the first and second nasal passages and joining the end regions. In use the dilator acts to stabilize and/or expand the nasal outer wall tissues and prevent said tissues from drawing inward during breathing.

Embodiments of the nasal dilator of the present invention include, without limitation, new and non-obvious means to direct the resilient properties thereof. Said means include one or more material separations, or discontinuity of shape of material, formed within the peripheral edges of the truss (an interior material separation), and may include one or more material separations or discontinuity of shape of material extending inward from a peripheral edge of the truss (an exterior material separation). Said material separations may be formed before, during or after the peripheral shape of the dilator is die cut from the aforementioned continuous laminate of materials. An interior material separation may also include forming, modifying or configuring at least a portion of the resilient layer before assembling the constituent layers of the dilator into the vertical laminate. Said formation, modification or configuration may include forming the peripheral shape of the resilient member, such as gradiently tapering its width, or may include forming component extensions such as spring fingers, or may include interior or exterior material separations, such as a cut, opening or notch, as described above with respect to the truss, but made to the resilient member alone.

An interior material separation may form a flap capable of separating or vertically protruding, in part, from the truss when the dilator is flexed across the nose of a wearer. Similarly, an exterior material separation may form a horizontal protrusion, also capable of separating, in part, from the truss when the dilator is flexed across the nose of a wearer. In either case, said separation or vertical protrusion changes the angle of focused spring biasing forces, at least in part, and thus shifts or transforms at least some of said forces from primarily peel and tensile forces to primarily shear forces. Said change in angle further redistributes or imparts said transformed forces to tissue engaging surface areas extending beyond the material separation. Thus, spring biasing forces may be distributed to the potentially larger surface area of the dilator end regions, as opposed to a greater delaminating tendency, such as that from peel forces, being imparted to a smaller surface area. Said potential larger surface area is as a result of the configuration of the end regions of the truss and/or the configuration of the respective layers of the dilator. The effect of material separations can lessen overall delaminating forces without reducing the spring biasing force of the dilator, in that shear forces are more easily withstood by the tissue engaging adhesives typically disposed on the tissue engaging surfaces of the dilator. Accordingly, a lesser amount of adhesive and/or less aggressive adhesive (and thus less costly) disposed on the tissue engaging surfaces of the dilator would, in addition, be more comfortable to the user and more easily removed from the tissue so engaged. An opposing pair of said material separation may be spaced apart along the longitudinal centerline of the truss.

An interior material separation extending vertically through the dilator, including the resilient layer, may also form a flap capable of separating or vertically protruding, in part, from the resilient layer. Said separation or vertical protrusion may also change, at least in part, the angle of spring biasing forces thereof, while allowing spring biasing forces to continue along a further extent of the resilient member or component. Said interior material separation may be confined within the peripheral edges of the resilient layer material or, alternatively, may sever the resilient member from one long edge thereof and extend across a portion of its width.

Means to direct resilient properties thus also include a dynamic relationship between the effects of interior and exterior material separations, including the degree of horizontal spacing between an opposing pair thereof, and any other modification to, or configuration of, the resilient layer, such as its peripheral shape or the inclusion of additional material separations made thereto.

The preferred embodiments of the present invention further include a truss with means for horizontally aligning the dilator to the nose of a wearer comprising a positioning aid located at the intermediate region forming a separation, projection or other index marker; means to spread the spring biasing force of resilient layer to a greater, primarily lateral, surface area of dilator, and means to prevent one or more material separations from separating in part from the truss. This latter means may also be used to extend or increase the tissue engaging surface area of the truss.

The skilled man in the art will appreciate the applicability of the continually developing art of medical device converting; specifically, continuous rotary laminating and die cutting, and flatbed and class A tool die cutting and punching.

The present invention is not limited to the illustrated or described embodiments as these are intended to assist the reader in understanding the subject matter of the invention. The preferred embodiments are examples of forms of the invention comprehended by the devices taught, enabled, described, illustrated and claimed herein. All structures and methods which embody similar functionality are intended to be covered hereby. In certain instances, the devices depicted, taught, enabled and disclosed herein represent families of new, useful and non-obvious tissue dilators having a variety of alternate embodiments. The skilled man will appreciate that features, devices, elements, members or components thereof, methods, processes or techniques may be applied, interchanged, eliminated in whole or part, or combined from one embodiment to another. Dilator members or components thereof, materials, layers or regions may be of differing size, area, thickness, length or shape than that illustrated or described while still remaining within the purview and scope of the present invention. The preferred embodiments include, without limitation, the following numbered, discrete forms of the invention, as more fully described below.

### BRIEF DESCRIPTION OF THE SEVERAL VIEWS OF THE DRAWING

In the drawings which accompany this disclosure, like elements are referred to with common reference numerals. Where there is a plurality of like objects in a single drawing figure corresponding to the same reference numeral or character, only a portion of said like objects may be identified. After initial description in the text, some reference characters may be placed in a subsequent drawing(s) in anticipation of a need to call repeated attention to the referenced object. Drawings are not rendered to scale.

FIG. 1 is an exploded perspective view of a nasal dilator in accordance with the present invention.

FIG. 2 is a perspective view of the nasal dilator of FIG. 1.

FIG. 3 is a plan view of the nasal dilator of FIG. 1 including one and two resilient member variations thereof.

FIG. 4 is a fragmentary plan view, on an enlarged scale, illustrating one end region of a nasal dilator in accordance with the present invention.

FIG. 5 is a perspective view, on an enlarged scale, of the nasal dilator of FIG. 4 secured to a nose.

FIG. 6 is a front elevation view of the nasal dilator of FIG. 5 secured to the nose.

FIG. 7 is a is a perspective view, on an enlarged scale, of a dilator in accordance with the present invention secured to a nose.

FIG. 8 is a plan view of a variation of the nasal dilator of FIG. 1.

FIG. 9 is a plan view of a variation of the nasal dilator of FIG. 1.

FIG. 10 is a plan view of a variation of the nasal dilator of FIG. 1.

FIG. 11 is a plan view of an alternative form of nasal dilator embodying features of the present invention.

FIG. 12 is a fragmentary plan view, on an enlarged scale, illustrating one end region of the nasal dilator of FIG. 11.

FIG. 13 is a plan view of an alternative form of nasal dilator embodying features of the present invention.

FIG. 14 is a perspective view, on an enlarged scale, illustrating a nasal dilator in accordance with the present invention secured to a nose.

FIG. 15 is a fragmentary plan view, on an enlarged scale, illustrating an alternative end region structure to that of the nasal dilator of FIG. 14.

FIG. 16 is a plan view of an alternative form of nasal dilator embodying features of the present invention.

FIG. 17 is a fragmentary plan view, on an enlarged scale, illustrating an end region variation of the nasal dilator of FIG. 16.

FIG. 18a is a plan view of an alternative form of nasal dilator embodying features of the present invention. FIGS. 18b-18d are plan views thereof and FIGS. 18e-18f are exploded perspective views thereof.

FIG. 19a is a fragmentary plan view, on an enlarged scale, illustrating a portion of one end region of the nasal dilator of FIG. 18a. FIGS. 19b-19f are also fragmentary plan views of enlarged scale illustrating a portion of one end region of the nasal dilators of FIGS. 18b-18f, respectively.

FIG. 20 is a plan view of an alternative form of nasal dilator embodying features of the present invention.

FIG. 21 is a fragmentary plan view, on an enlarged scale, illustrating a portion of one end region of the nasal dilator of FIG. 20.

FIGS. 22-24 and 27-30 are plan views and FIG. 25 is a perspective view illustrating alternative forms of the dilator device depicted in FIGS. 20 and 21.

FIG. 26 is a fragmentary plan view on an enlarged scale of the dilator of FIG. 25.

FIGS. 31-34 are plan views illustrating a variation, in accordance with the present invention, of the dilator devices depicted in FIGS. 28-30 and 35, respectively.

FIGS. 35-37 are plan views illustrating a variation, in accordance with the present invention, of the dilator device depicted in FIGS. 20 and 21.

FIG. 38 is a plan view illustrating an alternative form of the dilator devices shown in FIGS. 35-37.

FIG. 39 is a plan view illustrating an alternative form of the dilator device shown in FIG. 18c.

FIG. 40 is a plan view of an alternative form of nasal dilator embodying features of the present invention.

FIG. 41 is a fragmentary plan view on an enlarged scale of the dilator of FIG. 40.

FIG. 42 is a plan view illustrating a variation of the nasal dilator of FIG. 40.

FIGS. 43-46, 48-49 and 51 are plan views of an alternative form of nasal dilator embodying features of the present invention found in FIGS. 18-20.

FIGS. 47, 50 and 52 are exploded perspective views of the nasal dilators depicted in FIGS. 46, 49 and 51 respectively.

FIGS. 53-55 and 57 are plan views illustrating examples of a variation, in accordance with the present invention, of the dilator device depicted in FIGS. 43-52.

FIG. 56 is an exploded perspective view of a variation of the nasal dilator of FIG. 55.

FIGS. 58-61 are plan views, including fragmentary plan views on an enlarged scale, illustrating examples of an alternative form of nasal dilator embodying features of the present invention found in FIGS. 18-20.

### DETAILED DESCRIPTION OF THE INVENTION

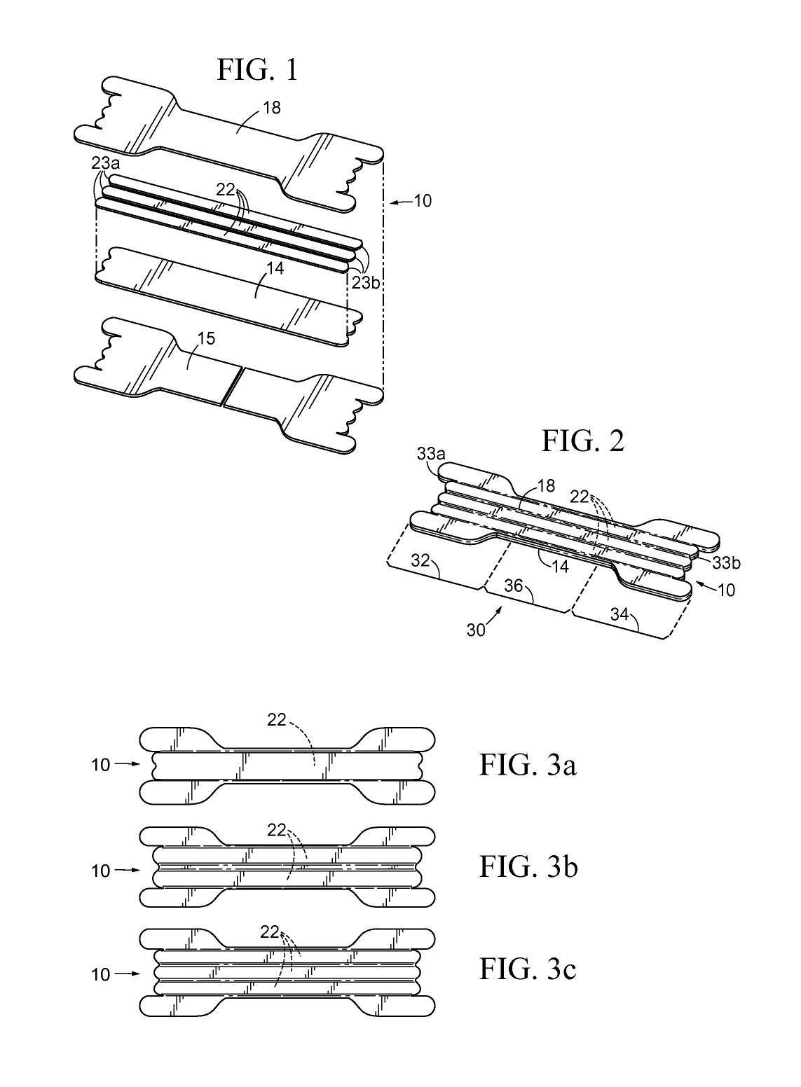

An embodiment of a nasal dilator, 10, in accordance with the present invention is illustrated in FIG. 1. Dilator 10 comprises a vertical laminate of material layers including: a base layer composed of at least one base member, 14, including components thereof; a resilient layer comprised of at least one resilient member, 22, including components thereof; and a cover layer composed of at least one cover member, 18, including components thereof. A protective layer of release paper liner, 15, removably covers any exposed adhesive from any layer preliminary to use of dilator 10 on the nose of a wearer. The periphery of release liner 15 may correspond to the periphery of dilator 10 or a periphery exceeding one or more dilators 10. The components or layers of dilator 10 are preferably aligned along their longitudinal centerlines.

The preferred material for the base and cover layers is from a group of widely available flexible nonwoven synthetic fabrics that allow the skin on user nose 11 to exchange gases with the atmosphere and to maximize comfort of dilator 10 thereon. Alternatively, any suitable fabric or plastic film may be used. A continuous pressure sensitive adhesive substance, biocompatible with external human tissue, is disposed on at least one flat surface side of said material which is the adhesive side, opposite the non-adhesive side. The non-adhesive side is typically opposite the skin engaging side. A protective layer of continuous release paper liner covers said adhesive. Said materials are typically available in continuous rolls wound in a machine direction (MD) or warp, which is perpendicular to the cross direction (XD) or fill, of the fabric. The base and cover layers of dilator 10 may be fabricated parallel to either the warp or the fill of said fabrics. The preferred material for the resilient layer is a biaxially oriented polyester resin, Poly(ethylene terephthalate), (PET or boPET). PET has suitable spring biasing properties both MD and XD, and is widely available as an industrial commodity under trade names such as Mylar® and Melinex®. PET comes in a variety of standard thickness including 0.005″, 0.007″, and 0.010″. Alternatively, any plastic film having the same or similar tensile, flexural, or elastic modulus values would also be suitable.

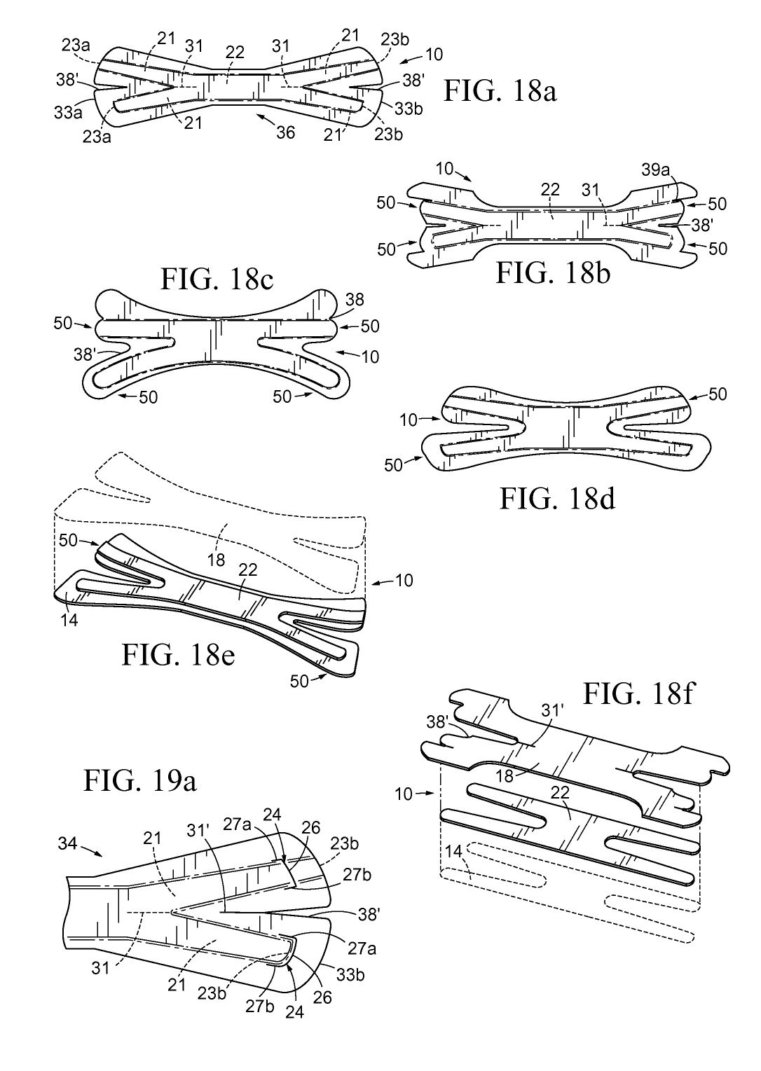

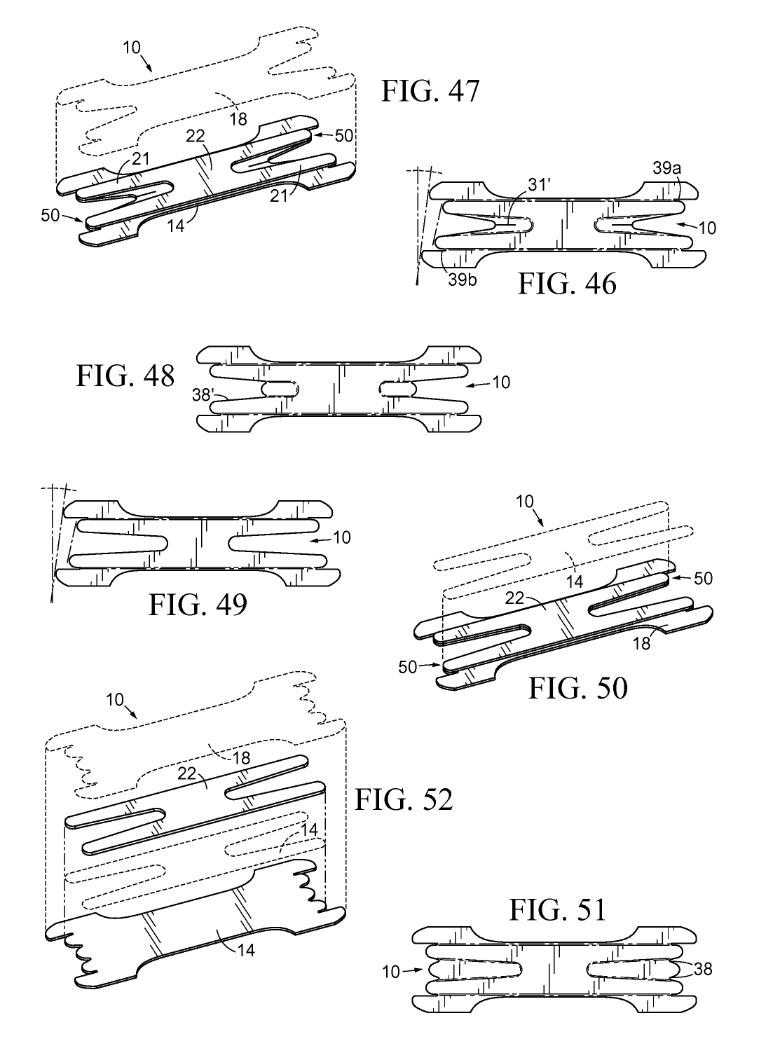

The width, length and peripheral outline or edges of dilator 10 may be defined by the base layer, cover layer, or a combination of any two or more layers or portions thereof. The base and cover layers of dilator 10 may have like or dissimilar dimensions or peripheral edges, in whole or in part, compared to each other. Their respective peripheral shapes may be uniform or non-uniform, and may also be of like or dissimilar size or scale. Portions of any layer may define a horizontal region of the dilator or a portion thereof. Furthermore, the base and cover layers of dilator 10 may be interchanged, or either the base layer or cover layer may be optionally eliminated in whole or in part. The base and resilient layers may have identical peripheral edges, and thus may be formed as a single unit. FIGS. 18e, 18f, 25, 40, 47, 50 and 52 depict cover member 18 and/or base layer 14 in dashed lines to exemplify these optional configurations.

Portions of one or both flat surfaces of any layer, member or component thereof, may overlap portions of any flat surface of another layer. Preferably, however, the base layer acts as a buffer in engaging the user's skin, as described hereinbefore with respect to medical devices, and portions of one or more dilator layers may engage nasal outer wall tissues simultaneously. When engaged on the nose of a wearer, preferably no portion of a layer extends substantially over a skin surface area beyond those surface areas associated with the nasal passages as described hereinbefore.

As illustrated in FIG. 2, the laminated layers of dilator 10 form a unitary, or single body, truss, 30, having horizontal regions as indicated by bracketed broken lines. Truss 30 includes a first end region, 32, a second end region, 34, and an intermediate region, 36, interconnecting first end region 32 to second end region 34. The layers, members or components of dilator 10 may overlap or extend from their originating region to an adjacent region. End regions 32 and 34 are adapted to engage outer wall tissues of first and second nasal passages, respectively.

The width of each end region is preferably greater than the width of respective portions or components of resilient member 22 extending horizontally therein. End regions 32 and 34 include lateral end edges, 33a and 33b, respectively, which define the outer, lateral ends of truss 30 and thus dilator 10. End edges 33a and 33b may be angled inward in a straight line between upper and lower corners of the long edges of dilator 10, said angle corresponding approximately to the line where the nose meets the cheek. The width of intermediate region 36 is preferably narrower than the width of end regions 32 and 34, preferably without resilient member 22 being formed narrower at its mid section that at its outer ends as a result.

Finished dilators 10 are typically die cut from a continuous laminate of material layers. However, dilator layers, members or components thereof, material separations or horizontal regions of truss 30 may be formed or die cut, in whole or part, from one or more continuous materials before, or during, assembly of the material laminate from which finished dilators 10 are die cut.

In fabricating dilators 10, end regions 32 and 34 are preferably formed as mirror images of each other. However, asymmetric or non-identical end region configurations have the advantage of providing disparate dilating forces and tissue engaging surface areas to opposing nasal outer wall tissues, and thus more accurate or customized dilation or stabilization to the respective nasal passages. It will thus be apparent to the skilled man in the art that virtually any two end region structures of the preferred embodiments herein may be intentionally combined in a given dilator device, as seen, for, example, in FIGS. 31-34, 37 and 57. For the sake of clarity and simplicity, however, most of the preferred embodiments illustrate end regions 32 and 34 as mirror images of each other. Additionally, certain of the enlarged fragmentary plan views refer to features of one truss end region, but are equally applicable to the opposing end region.

When engaged to and flexed across a nose 11, dilator 10, through its resilient means as a result of its constituent members and layers combined to form single body truss 30, acts to stabilize and/or expand the nasal outer wall tissues and prevent said tissues from drawing inward during breathing.

Dilator 10 includes resilient means having resilient properties provided through its resilient layer and configured to provide suitable spring return biasing force as described hereinbefore. Overall spring biasing force is generally determined by the width, length, and thickness of at least one resilient member 22 or the resilient layer as a whole from its constituent member(s) and/or components.

Resilient member 22 preferably has an adhesive substance disposed on at least a portion of at least one of two opposite flat surface sides for engaging or laminating it to other layers, members or components of dilator 10 or for engaging the skin surface of the nose. Resilient member 22 has opposite terminal ends, 23a and 23b, respectively, that may conform to at least portions of the lateral end edges 33a and 33b of dilator 10. Terminal ends 23a and 23b may extend to one or both of said lateral end edges of dilator 10, or may extend short of one or both end edges.

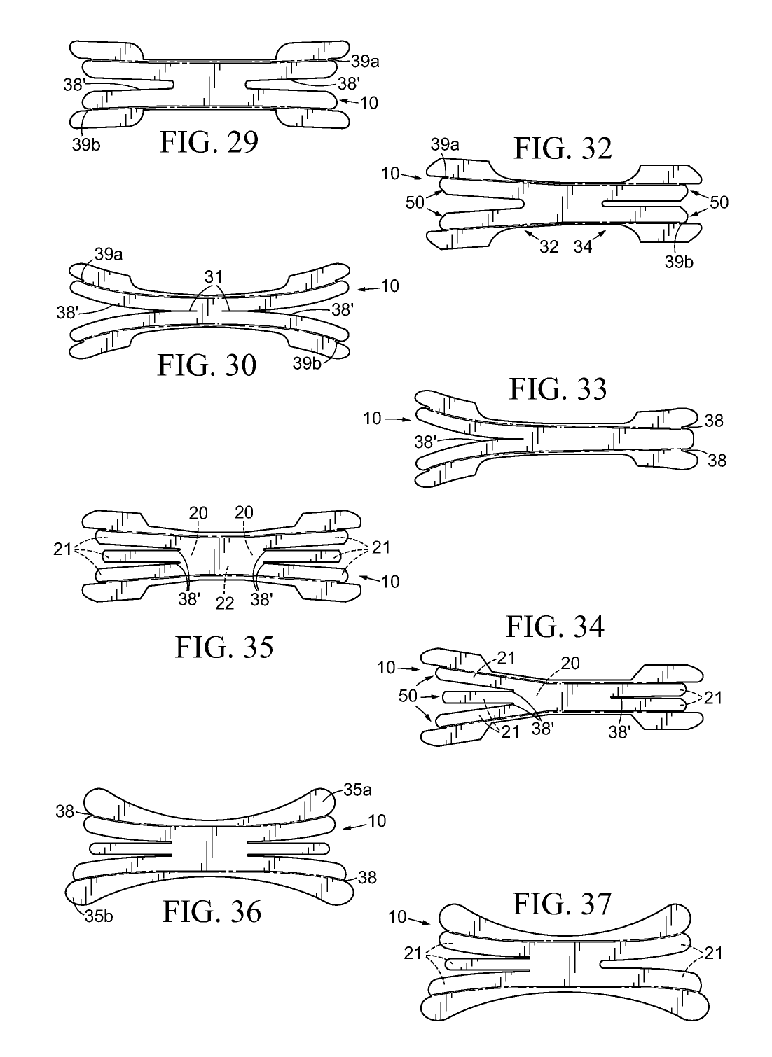

Dilator 10 includes means to direct its resilient properties. Said means may comprise configuration of, or modification to, the resilient layer or the material from which the resilient layer is formed. Said configuration or modification may be made either in the course of forming resilient member 22, or may be made to the resilient layer material separately, or at the time said material is assembled into the continuous material laminate from which dilator 10 is die cut (i.e., at the time the vertical laminate of dilator 10 is formed). Said configuration or modification may include cuts, notches, openings, or the like formed in the resilient layer material; or by varying the finished dimensions of the resilient member or a component thereof, such as by forming a gradiently tapered width; or by peripheral shape of the resilient member, such as by extensions or divergent spring finger components extending outward from its longitudinal extent, as seen, for example, in FIGS. 18-19, 20-30, and 31-60; or by more than one resilient member, as seen, for example, in FIGS. 1-2, 3b-3c, 4-10, and 13-15, with each member contributing a portion of the total spring biasing force. Having divergent spring fingers or multiple resilient members may increase the effective surface area subject to resilient layer spring biasing forces by spreading those forces to a greater, primarily lateral, surface area of dilator 10.

Said means to direct the resilient properties of dilator 10 further comprises at least one separation or discontinuity of shape of material of one or more regions or layers of truss 30. Said material separation or discontinuity of shape comprises a relief cut or back cut, slit, opening, notch, or the like, having a lateral and/or longitudinal extent, formed within the peripheral edges of dilator 10 (an interior material separation), or extending inward from a peripheral edge thereof (an exterior material separation). Said material separation extends vertically through at least one layer of dilator 10 and may optionally extend through release liner 15.

An interior material separation extending across the width of the resilient member 22 redefines its functional length (said function being the creation of spring biasing forces when flexed), and thus changes the dimensional relationship between its length and width/thickness. This also changes the spatial, dimensional relationship between the functional portion of the resilient layer and the other members or layers of dilator 10. Said interior material separation thus further creates and defines at least one additional, substantially nonfunctional, component of the resilient layer.

One or more opposing pairs of interior or exterior material separations may be placed within or near respective end regions 32 and 34 of truss 30. An opposing pair is preferably positioned in a spaced apart relationship along or near the dilator's longitudinal centerline as seen, for example, in FIGS. 11-17, and 19. The spacing apart of a pair of material separations is dynamic, and determines, at least in part, some degree of direction of resilient properties, as well as the longitudinal extent of dilator 10 affected thereby. Said means to direct resilient properties thus further comprises a dynamic relationship between the effect of an opposing pair of material separations and any other modification to, or configuration of, the resilient layer, including additional material separations or pairs thereof.

For the sake of clarity and simplicity, interior and exterior material separations are shown uniform or as mirror images of each other in the preferred embodiments illustrated herein. As previously noted, however, asymmetric or non-identical elemental configurations have the advantage of providing disparate dilating forces and tissue engaging surface areas to opposing nasal outer wall tissues, and thus more accurate or customized dilation or stabilization to the respective nasal passages. Accordingly, it will be apparent to the skilled man that disparate material separations may be intentionally combined in a given dilator device, or identical or opposing material separations may be of dissimilar size or scale. Additionally, certain of the enlarged fragmentary plan views illustrate material separations positioned at one end region, but are equally applicable to the opposing end region.

As detailed hereinbefore, an interior material separation extending vertically through dilator 10, including the resilient layer, may be contained entirely within the peripheral edges of resilient member 22 (or a component thereof) or extend inward from a peripheral edge thereof. Said material separation may allow formation of a flap capable of separating or vertically protruding, in part, from the resilient layer. Said separation or vertical protrusion may also change, at least in part, the angle of spring biasing forces of resilient member 22, while also allowing spring biasing forces to continue along a further extent of the resilient member or component. By virtue of extending vertically through the resilient member without severing its entire width, said interior material separation reduces the total spring biasing force of resilient member 22, primarily from the point of said separation to the adjacent terminal end thereof. In this manner, an opposing pair of interior material separations may be spaced apart along the horizontal extent of resilient member 22 so as to redirect a greater portion of total spring biasing force between the spaced apart pair and a corresponding lesser portion extending from each separation to corresponding terminal ends 23a and 23b.

Accordingly, the type, number, and location of one or more interior and/or material separations or pairs thereof, the configuration of resilient member 22 and its corresponding resiliency, the relative size and shape of end regions 32 and 34, and the dynamic relationships between these various elements, all contribute to directing the resilient properties of dilator 10. Various examples thereof are given in the preferred embodiments and discussed in more detail below.

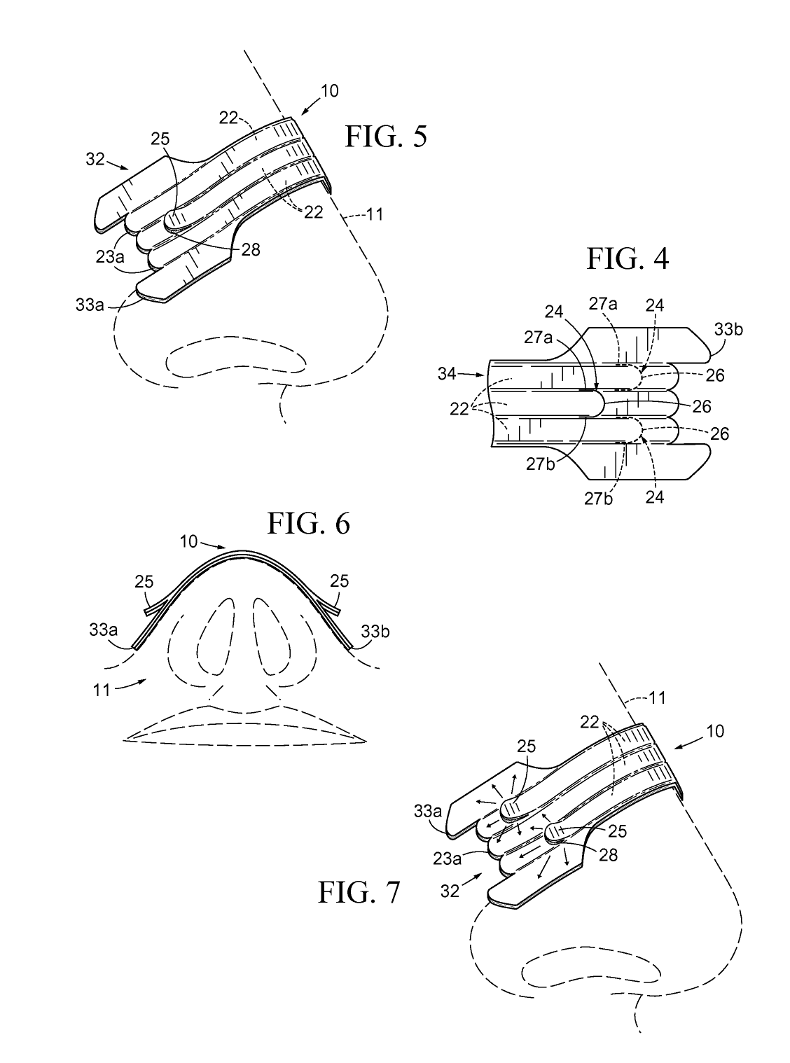

As more clearly seen in the plan views of FIGS. 4, 11-13, 15, and 19, an interior material separation comprises a relief cut, 24, located within each end region of truss 30. Relief cut 24 preferably extends vertically through the cover, resilient and base layers of dilator 10. FIG. 4 more particularly identifies relief cut 24 having an outside edge, 26, which defines its width, and upper and lower long edges, 27a and 27b which define at least portions of its length. Outside edge 26 preferably corresponds to at least a portion of the nearest end edge 33a or 33b, respectively, of end regions 32 and 34. (As discussed hereinbefore, each end region is shown as a mirror image of the other, so only one end region will be described with particularity.) Outside edge 26 severs the entire width of resilient member 22 laterally, preferably extending slightly past the upper and lower long edges thereof, before turning to upper and lower edges 27a and 27b. Upper and lower edges 27a and 27b extend inward preferably about 0.125″ in a direction parallel to upper and lower long edges of resilient member 22. Relief cuts 24 redefine the functional length of resilient member 22, as described hereinbefore, creating additional, substantially nonfunctional, resilient layer components. FIGS. 4, 13, and 15 illustrate further examples of positioning at least one relief cut 24 in an end region where the dilator includes multiple resilient members. In these examples, as in FIGS. 5, 7 and 14, three substantially parallel resilient members are shown.

FIGS. 5-7 and 14 show dilator 10 adhered to and flexed across the bridge of a nose, 11. Relief cut 24 allows formation of a flap, 25, at the redefined terminal ends of resilient member 22, said flap capable of separating or vertically protruding, in part, from respective end regions 32 and 34 of truss 30, and leaving a corresponding opening or gap, 28, from where it separates from the truss when dilator 10 is engaged to nose 11. The length of upper and lower edges 27a and 27b and the width of outside edge 26 of relief cut 24 define the shape and dimensions of flap 25; its length being parallel to the longitudinal extent of resilient member 22. Said length determines in part the degree of said separation or vertical protrusion and the corresponding change in angle, and thus transfer, of focused spring biasing forces from primarily peel forces and tensile forces into primarily shear forces, as discussed hereinbefore. Said transformed spring biasing forces are redistributed or imparted to tissue engaging surfaces of dilator 10 extending in an area between gap 28 and the surrounding peripheral edges of end region 32, as generally illustrated by directional arrows in FIG. 7. Relief cuts 24 are preferably spaced apart along the longitudinal extent of dilator 10, placed closer to respective end edges 33a and 33b than to intermediate region 36, so as to direct resilient properties along a greater, rather than lesser, longitudinal extent of dilator 10.

FIGS. 11-12 illustrate an alternative structure of relief cut 24 in which outside edge 26 forms a scalloped edge identical to a portion of corresponding end edge 33b or 33b. As more particularly illustrated in FIG. 12, outside edge 26 of relief cut 24 extends from upper and lower edges 27a and 27b, preferably intersecting upper and lower long edges of resilient member 22 at right angles thereto before forming a single scalloped edge. Said scalloped edge conforms to a corresponding center portion of end edge 33b of end region 34. The total length of relief cut 24, denoted by bracketed broken lines, is defined by the length of upper and lower edges 27a and 27b, plus the horizontal extent of the scalloped portion of outside edge 26 extending toward end edge 33b.

FIG. 12 further illustrates end edge 33b having three portions situated along a common lateral plane, represented by broken lines. Said lateral plane may be optionally set at an oblique angle to the long axis of dilator 10, corresponding approximately to the line where nose 11 meets the cheek of a face 12. The upper and lower of said three portions curve arcuately inward from the outside corners of upper and lower long edges of end region 34, forming an exterior material separation comprising a valley, 38, at the intersections of respective upper and lower corners of terminal end 23b of resilient member 22. From the intersections formed by valleys 38, end edge 33b curves outwardly again to form said scalloped center portion. The apex of said center portion corresponds to the longitudinal axis of resilient member 22, with terminal end 23b thereof terminating along said scalloped center portion.

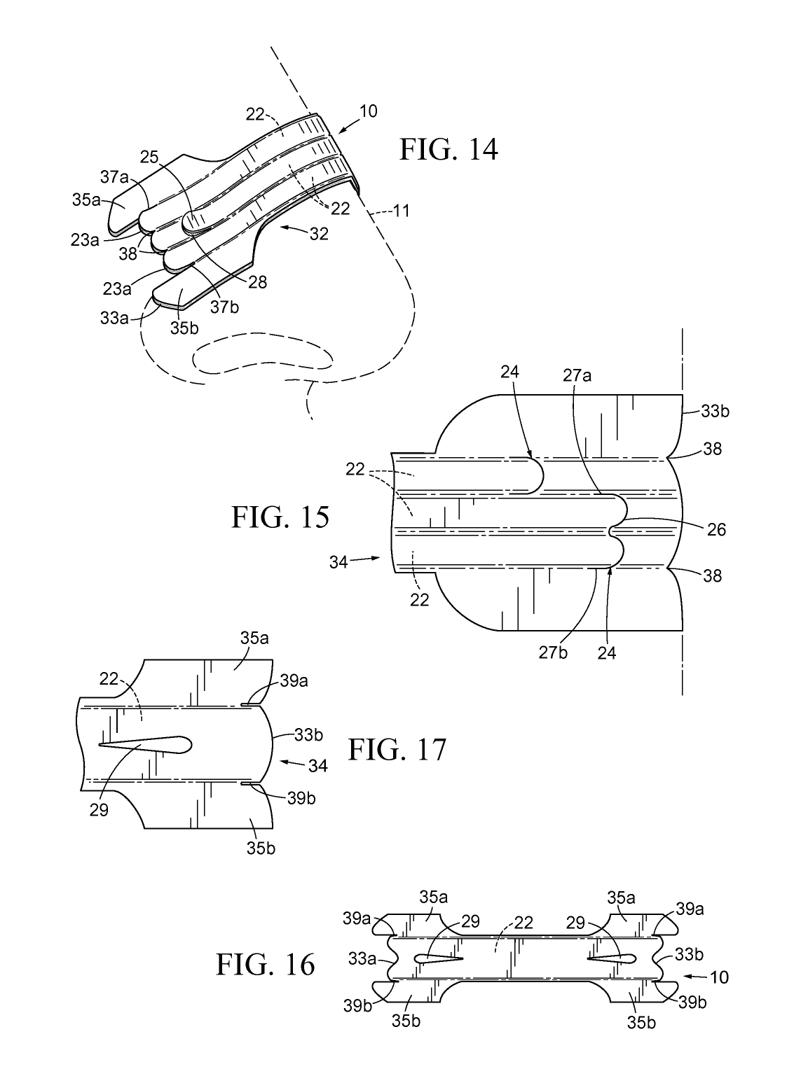

FIGS. 13-14 illustrate a combination of interior and exterior material separations in accordance with the present invention. FIG. 13 shows end edges 33a and 33b having scalloped portions which correspond substantially to terminal ends 23a and 23b of parallel, spaced apart resilient members 22. Said terminal ends define the longitudinal extent of dilator 10. A pair of interior material separations comprising relief cuts 24 are placed in a spaced apart relationship in opposing end regions of truss 30, each relief cut forming a scalloped edge across the width of at least one of said resilient members 22. FIG. 13 shows outside edge 26 of relief cut 24 intersecting upper and lower long edges 27a and 27b, respectively, at oblique angles thereto. The shape of outside edge 26 preferably corresponds to a corresponding portion of scalloped end edge 33b.

FIGS. 13 and 14 further illustrate a pair of exterior material separations comprising upper and lower back cuts, 37a and 37b, extending vertically through at least the cover layer of dilator 10 and inward from end edge 33b. Each back cut is positioned at the intersection of a corresponding valley 38, adjacent and parallel to the upper and lower long edges of at least one resilient member 22. This arrangement defines a horizontal protrusion at the end portions of said one resilient member. Lower back cut 37b forms a separation between said horizontal protrusion and a corresponding lower extension, 35b. Extension 35b may optionally extend horizontally beyond terminal end 23b, as seen in FIG. 14, and thus may further define the longitudinal extent of dilator 10.

The interior material separation positioned in end region 32 or 34 may allow formation of a flap 25 at the redefined terminal ends of upper resilient member 22, capable of separating or vertically protruding, in part, from truss 30 when dilator 10 is flexed across the nose. Similarly, the horizontal protrusion defined by upper and lower back cuts 37a and 37b is also capable of separating or protruding vertically, in part, from the truss when the dilator is flexed across the nose of a wearer, as particularly seen in FIG. 14. In each case the material separation changes the angle, in part, of focused spring biasing forces, transforming said forces as described hereinbefore. With respect to the interior material separation, said transformed forces are imparted to the end region in general, as indicated previously by directional arrows in FIG. 7. With respect to said exterior material separations, said transformed forces are imparted, at least in part, to extensions 35a and 35b.

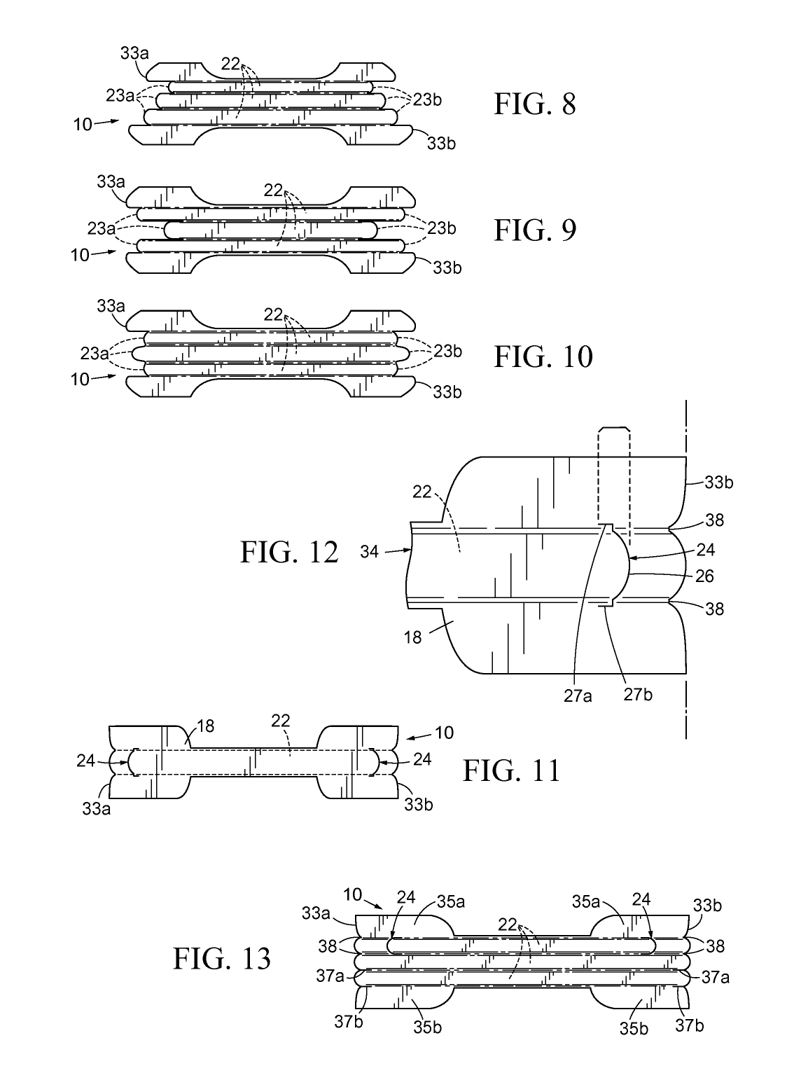

The parallel spaced apart resilient members 22 may be of like or dissimilar width, as illustrated previously with regard to FIGS. 8-10. A dynamic relationship exists not only between the respective spring biasing properties of multiple resilient members of dissimilar widths, but also between the location of relief cuts 24, the length(s) of relief cut(s) 24, back cuts 37a and 37b, and the combined spring biasing forces generated by said resilient members 22. Though the relief cuts and back cuts are shown as symmetric pairs, it will be apparent to the skilled man that these elements may be resized, recombined or omitted.

FIG. 15 illustrates an alternative end region structure to that shown in the embodiment of FIG. 14, in which relief cut 24 extends across a pair of resilient members 22. Respective scalloped portions of outside edge 26 extend across the width of each resilient member. End edge 33b has three portions situated along a common lateral plane. Said plane is shown perpendicular to the long axis of dilator 10, but may be optionally situated at an oblique angle thereto, corresponding approximately to the line where the nose meets the cheek. The upper and lower of said three portions curve arcuately inward from the outside corners of upper and lower long edges of end region 34, forming valley 38 at intersections adjacent above and below respective upper and lower corners of terminal ends 23b of said pair of resilient members 22. From said intersections end edge 33b curves outwardly again to form said scalloped center portion. The apex of said center portion corresponds to the longitudinal axis of the pair of resilient members 22, with terminal ends 23b thereof terminating along said scalloped center portion.

FIGS. 16-17 illustrate another combination of interior and exterior material separations in accordance with the present invention. End edges 33a and 33b form a scalloped portion corresponding to respective terminal ends 23a and 23b of resilient member 22. The distance between said terminal ends represents the longitudinal extent of dilator 10. Exterior material separations comprising upper and lower notches, 39a and 39b, are positioned parallel to and adjacent upper and lower long edges of resilient member 22. Notches 39a and 39b extend vertically through the base and cover layers of dilator 10, and inward from end edges 33a and 33b, respectively. Notches 39a and 39b define intersections between said scalloped portion of end edges 33a and 33b and upper and lower tab extensions 35a and 35b, respectively, of end regions 32 and 34. Tab extensions 35a and 35b may optionally extend to, or beyond, said scalloped portions as shown in FIG. 16, the latter thus further defining the longitudinal extent of dilator 10.

As more particularly illustrated in FIG. 17, said scalloped mid portion of end edge 33b and notches 39a and 39b define a horizontal protrusion, also capable of separating in part from the end region 34, as discussed hereinbefore. Said separation changes the angle, at least in part, of spring biasing forces, and shifts and transforms said forces, similarly as described with respect to FIG. 13, imparting said transformed forces to both upper and lower tab extensions 35a and 35b of end region 34.

The dilator of FIGS. 16-17 further includes an opposing pair of interior material separations each comprising an elongated opening, 29, extending vertically through at least resilient member 22 and contained within the width thereof. Opening 29 may be optionally formed before assembly of the vertical laminate of dilator 10, or (as shown) formed as dilator 10 is die cut from a continuous material laminate. Opening 29 has a gradient increase in width along its length, extending horizontally from inward to outward, which defines corresponding adjacent upper and lower portions of resilient member 22 having a gradient reduction in width. Opening 29 may be of any size or shape contained within the width of resilient member 22. Each of the opposing pair thereof is preferably positioned horizontally between the lateral centerline of truss 30 and respective end edges 33a and 33b.

The relative width of opening 29 compared to the width of resilient member 22 thereat, together with the distance between said opposing pair of openings 29 defines a dynamic relationship, which determines spring biasing forces generated between said openings and extending beyond each opening to corresponding terminal ends 23a and 23b, respectively, of resilient member 22. Another dynamic relationship exists between the configuration of interior material separations, openings 29, and the exterior material separations at respective end edges 33a and 33b.

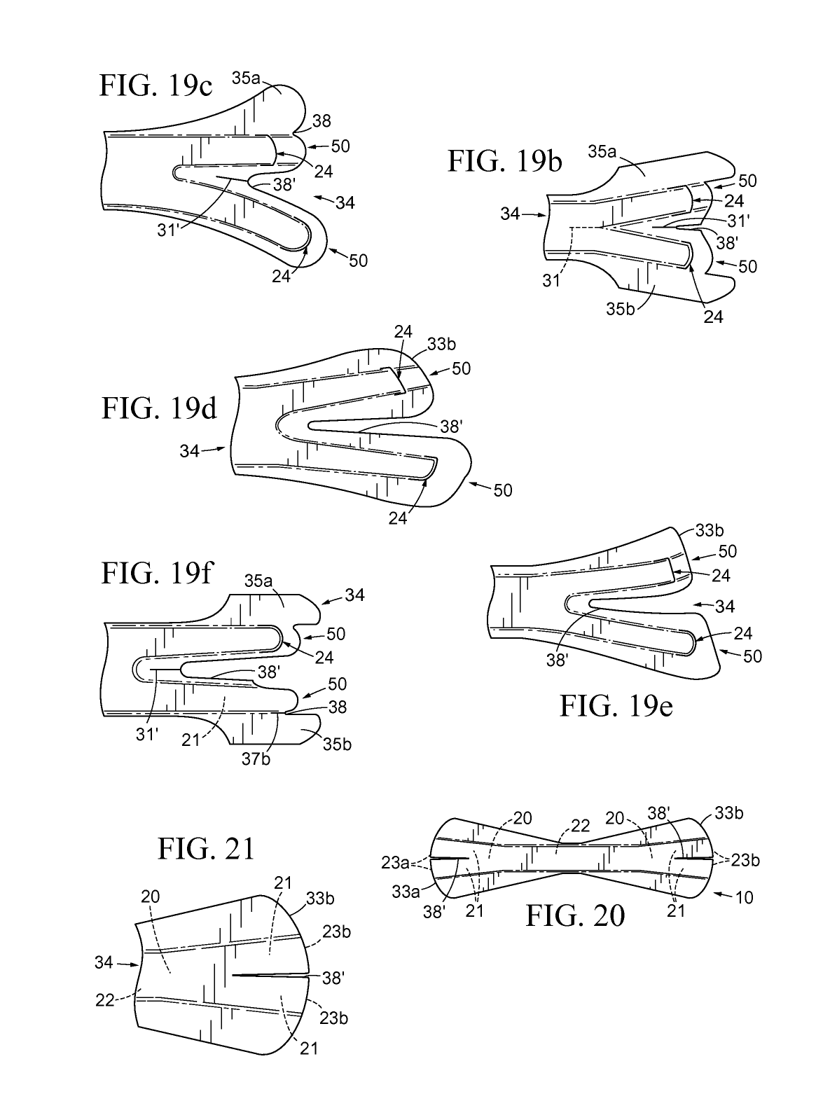

FIGS. 18-19 illustrate an embodiment of dilator 10 in accordance with the present invention in which the end regions of truss 30 include upper and lower bifurcated end region portions. In addition, resilient member 22 includes a plurality of component spring fingers, 21, diverging and extending outward from a common center. Said common center is preferably aligned with the lateral and longitudinal centerlines of intermediate region 36.

Spring biasing forces generated by the resilient layer of dilator 10 are gradiently reduced, at least in part, in the course of being directed to spring fingers 21. Upper and lower fingers 21 have uniform gradient widths, but may optionally curve, be asymmetric, and may be equidistant or of varying distance from said common center. As noted hereinbefore, divergent or asymmetric dilator features can provide disparate spring biasing forces. Fingers 21 may be further defined by a slit, 31, extending inward from the point where upper fingers diverge from lower fingers as seen, for example, in FIGS. 18a-18b and corresponding FIGS. 19a-19b.

Fingers 21 extend into corresponding bifurcated portions of end regions 32 and 34. Terminal ends 23a and 23b of upper fingers 21 extend to, and conform with, portions of end edges 33a and 33b thereat. Terminal ends 23a and 23b of lower fingers 21 extend short of end edges 33a and 33b. However, it will be apparent to the skilled artisan that the lower spring fingers may extend to the dilator end edges instead of the upper spring fingers, as illustrated, for example, in FIGS. 18f/19f. Alternatively, the dilator may be rotated 180 degrees in use so that the upper spring finger effectively become lower spring fingers.

Spring fingers 21 and slits 31 of resilient member 22 are configurations preferably made prior to assembling the vertical laminate of dilator 10. The divergent extent of spring fingers 21 determines the lateral spread of spring biasing forces at end regions 32 and 34. The gradient width and the length of each spring finger 21, defined in part by the length of slit 31, determines the gradient reduction in spring biasing forces along the longitudinal and lateral extents of resilient member 22. In addition, the divergent end region structure of dilator 10 provides additional lateral, torsional, flexibility primarily at the end regions, allowing dilator 10 to simultaneously effect dilation of nasal outer wall tissues adjacent both the nasal valve and nasal vestibule.

As further seen in FIG. 18, and more particularly illustrated in FIG. 19, end edge 33b has one of two exterior material separations comprising a valley, 38′, forming the intersection between said upper and lower bifurcated end region portions. In FIGS. 19a-19c and 19f, a second exterior material separation comprising a slit, 31′, extends inward from the terminus of valley 38′ preferably along the longitudinal axis of truss 30, corresponding to slit 31 in resilient member 22. Slit 31′ preferably extends short of resilient member 22.

Where dilator 10 includes a plurality of spring fingers extending from a common center, as described herein, each spring finger 21 may be seen as terminating at a discrete engagement contact point, 50. Contact point 50 may include tissue engaging surface area of dilator 10 extending around or adjacent the spring finger end portion. Dilator 10 may be configured such that contact points 50 engage the tissues associated with the nasal passages at specific locations, for example: skin surfaces overlaying the nasal valve, nostril and nasal vestibule, respectively, as described hereinbefore, and/or skin surfaces above and outward from the nasal valve.

As further illustrated in FIG. 19, upper bifurcated end region portions include relief cut 24 extending across the width of upper spring finger 21. Another relief cut 24 is positioned outboard and adjacent terminal end 23b of lower spring finger 21, corresponding to the shape of said terminal end. Relief cuts 24 have upper and lower edges 27a and 27b, respectively, defining their length and extending parallel to the long edges of spring finger 21. Outside edge 26 preferably extends beyond upper and lower long edges of spring finger 21, substantially following the contour of the corresponding end edge 33b. Relief cuts 24 allow formation of a flap, as described hereinbefore, capable of separating or vertically protruding, in part, when dilator 10 is flexed across the nose; said separations or vertical protrusions changing the angle, in part, of spring biasing forces, as described hereinbefore, transforming said forces and imparting them, at least in part, to tissue engaging surface areas extending outward to corresponding peripheral edges of said bifurcated end region portions of truss 30.

FIGS. 18 and 19 further illustrate that lower spring finger terminal ends may extend beyond the upper finger terminal ends, or vice versa, and that redefined terminal ends formed by relief cut 24 may alter that relationship. FIGS. 18f and 19f show a portion of an inside long edge of valley 38′ merging with and defining a portion of the inside edge of lower spring finger 21 near where the end portion thereof extends to and conforms with the end edge of the truss. FIGS. 18f and 19f also illustrate that resilient member 22 may optionally have substantially parallel upper and lower long edges.

FIGS. 20-21 illustrate an embodiment in accordance with the present invention where enlarged end portions, 20, of resilient member 22, formed as a modification prior to assembling the vertical laminate of dilator 10, correspond generally to the shape of end regions 32 and 34 of truss 30. Resilient member end edges 23a and 23b extend to, and conform with, portions of end region end edges 33a and 33b, respectively. As more particularly illustrated in FIG. 21, valley 38′ extends inward from said end edge, simultaneously bifurcating end regions 32 and 34, as well as enlarged end portions, 20, of resilient member 22. Said bifurcation forms spring fingers 21 in resilient member 22 and upper/lower bifurcated end region portions having common inside long edges therewith.

Valley 38′ may be configured to gradiently reduce the width of at least one spring finger 21. Depending upon the dimensional relationship between the width of enlarged end portions 20 and the length and width of valley 38′, said bifurcation may laterally spread and/or reduce or gradiently reduce the spring biasing forces of dilator 10 primarily at end regions 32 and 34. This divergent end region structure provides additional lateral torsional flexibility primarily at the end regions of truss 30, allowing dilator 10 to simultaneously effect dilation of nasal outer wall tissues adjacent both the nasal valve and nasal vestibule.

It will be apparent to the skilled person in the art that the resilient member of dilator 10, including any spring finger components, is designed to exert a spring biasing force in a direction perpendicular to its longitudinal surface plane. It may be further apparent to skilled persons familiar with the preferred resilient layer material or equivalent thereof that the properties of this material renders spring fingers 21 incapable of flexing or exerting a tensing force in a direction parallel to the surface plane of the resilient member. That is, the spring fingers may not be pinched together or spread apart laterally without buckling longitudinally. Since resilient member 22 is secured, at least in part, to at least one of a base layer or cover layer, buckling would compromise engagement of the dilator to the skin of the nose. Furthermore, being secured to at least one of a base layer or cover layer, would, in itself, inhibit or wholly prevent movement of the spring fingers across said surface plane.

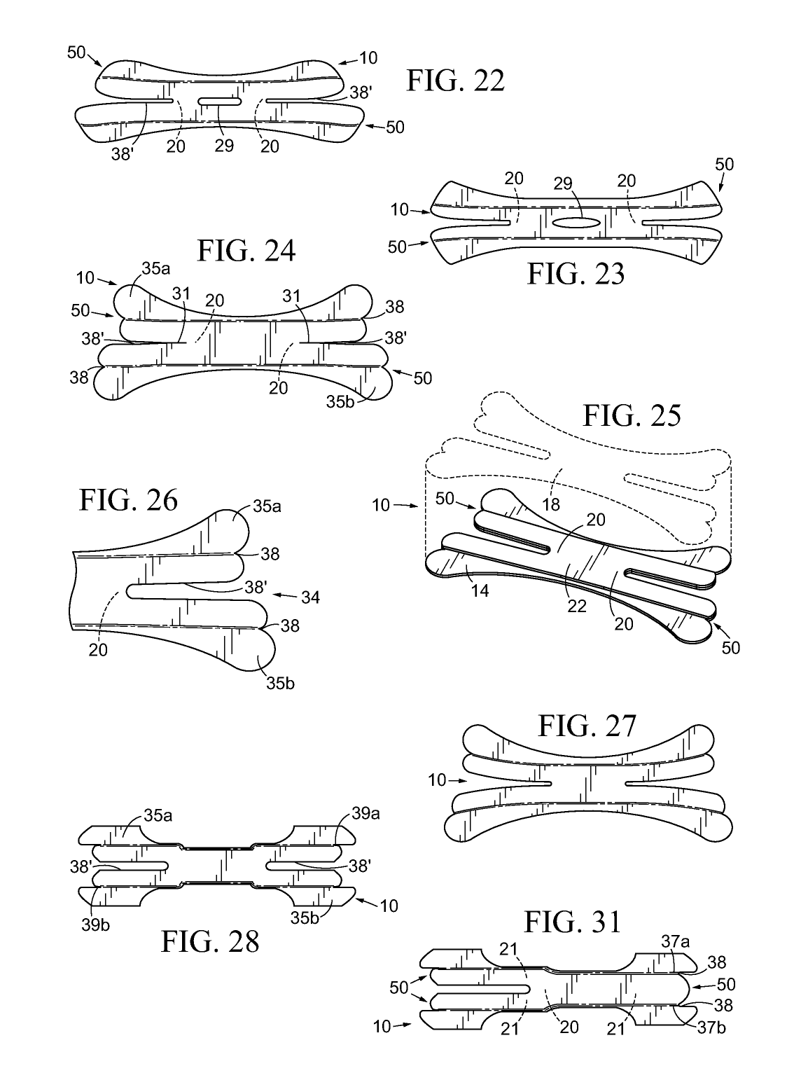

FIGS. 22-30 illustrate further examples of dilator 10 as described with regard to FIGS. 20-21. As seen in FIGS. 24-26, end portions 20 are only slightly enlarged laterally by virtue that the upper and lower long edges of resilient member 22 are only slightly farther apart at the truss end edges compared to the truss intermediate region. It will thus be apparent to the skilled person in the art that said upper and lower long edges may be substantially parallel, as seen previously in FIG. 18f, and further seen, for example, in FIGS. 28-29 and 43-57.

Dilator 10 as seen in FIGS. 24-30 include upper and lower tab extensions 35a and 35b, as described hereinbefore, and may further include upper and/or lower back cuts 37, corresponding valley 38, or upper and/or lower notches 39, which may be associated with tab extensions 35 as described hereinbefore.

As seen in FIGS. 22, 23, and 42, a single interior material separation comprising elongated opening 29 is positioned at the lateral and longitudinal centerlines of the truss, extending vertically at least through resilient member 22. Opening 29 also serves as an aid for aligning dilator 10 to the bridge of nose 11. The peripheral shape of resilient member 22 and opening 29 may be formed prior to assembling the constituent layers of dilator 10.

Opening 29 effectively reduces the spring biasing strength of the resilient member from that which would otherwise be generated. Accordingly, there is a dynamic relationship between the size of opening 29 and the dimensions of resilient member 22, said dynamic relationship contributing to the direction of spring biasing properties of dilator 10 as described hereinbefore.

FIGS. 31-37 illustrate further examples of material separations and spring finger end region configurations described previously with regard to FIGS. 20-21 and 22-30.

As seen in FIGS. 31-34, resilient member enlarged end portion 20 is formed in only one end region of the truss. The opposing end portion of the resilient member and surrounding end region thereat are substantially un-enlarged by comparison. Thus dilator 10 features asymmetric or non-identical end region configurations, which may provide disparate dilating forces and tissue engaging surface areas to opposing nasal outer wall tissues as described hereinbefore. End region 32 is shown in the drawing figures as the wider and end region 34 the narrower, however, it will be apparent to the skilled person in the art that that arrangement may be reversed.

Valley 38′ bifurcates enlarged end portion 20 of one end region so as to form at least two spring fingers 21. As seen in the drawing figures, the degree of lateral divergence between spring fingers, and the longitudinal extent of the spring fingers 21 and valley 38′ may be greater or lesser. Dilator 10 as seen in FIGS. 31 and 33 features two spring fingers in one bifurcated end region with the opposing un-bifurcated end region having one spring finger, for a total of three spring fingers extending from a common center. Valley 38′ may also bifurcate the un-enlarged end region as seen, for example, in FIGS. 32 and 34. Dilator 10 as depicted in FIG. 34 has three spring fingers corresponding to enlarged end portion 20 in one end region and two spring fingers extending into the opposing un-enlarged end region.

FIGS. 34-37 illustrate that a plurality of substantially similar valleys 38′ may extend inward from an end edge of the truss. Valleys 38′ separate enlarged end portion 20 of resilient member 22 into upper and lower spring fingers 21 and at least one middle spring finger interposed therebetween. Valleys 38′ may be seen as trifurcating an end region to form three spring fingers, however, the middle finger effectively separates the end region into upper and lower portions each having one spring finger adjacent a tissue engaging portion such as tab extension 35. In that latter sense the end region may still be seen as bifurcated.

In those embodiments wherein dilator 10 includes a plurality of spring finger components extending from a common center into an end region, the cumulative width of the spring fingers may be roughly equivalent to, but preferably not significantly greater than, the width of the common center from which the fingers extend.

FIG. 37 further illustrates that dilator 10 may include asymmetric resilient member end portions within a substantially symmetric overall periphery of the truss. Each end region is substantially identical in width, length, long edges, and having substantially identical tab extensions 35. However, a first plurality of spring fingers extends into one end region and a second plurality of spring fingers extends into the opposite end region. Again, the advantage being that of providing disparate dilating forces to opposing nasal outer wall tissues.

Dilator 10 of FIGS. 38-61 incorporate material separations and spring finger end region configurations as described with regard to either or both of FIGS. 18-19 and/or 20-21. Accordingly, exterior material separation valley 38′ is briefly recapped here:
As described with regard FIGS. 18-19, valley 38′, and optionally slit 31′, extends inward from an end edge of the truss through the base layer and/or cover layers of dilator 10, interposed between spring finger long edges, to form bifurcated end region portions. Spring fingers 21 and slits 31 of resilient member 22 are thus formed before assembling the vertical laminate.
As described with regard FIGS. 20-21, valley 38′ extends inward from an end edge of the truss, bifurcating end regions 32 and 34 and enlarged resilient member end portions 20, forming spring fingers and upper/lower bifurcated end region portions having common inside long edges. Valley 38′ and slit 31 thus extends through the base and/or cover layers as well as the resilient layer of dilator 10, and are preferably formed after assembling the vertical laminate.

Continuing now with FIG. 38, resilient member 22 includes enlarged end portions 20 and three spring fingers 21 extending into each end region of the truss. The middle fingers extend to and conform with portions of the end edges thereof, and the upper and lower spring fingers extend short thereof. Spring fingers may be further defined by slits 31 as described hereinbefore.

FIGS. 38 and 39 illustrate that the base or cover layer of dilator 10 may extend between the terminal ends of upper and lower spring fingers so as to have more material to engage the skin surfaces thereat. Optionally, exterior material separations extending inward from an end edge to bifurcate the end region may be omitted. However, FIG. 38 shows that valleys 38′ may include slits 31′ extending into the base and/or cover layers of dilator 10 in between spring finger long edges to effectively trifurcate each end region.

As seen in FIG. 39, the truss may include valleys 38 and back cuts 37a/37b to form a horizontal protrusion at each end of the upper spring fingers, capable of separating or protruding vertically, in part, from the truss when the dilator is flexed across the nose, as described hereinbefore. Like the middle spring fingers shown in FIG. 38, the upper spring fingers seen in FIG. 39 are substantially parallel to the longitudinal centerline of the truss, the adjacent spring fingers diverging arcuately therefrom.

FIGS. 40-41 illustrates that valley 38′ may optionally extend to about the point where upper spring fingers diverge from lower spring fingers, merging with and defining a portion of spring finger inside edges thereat. The base and/or cover layers may thus extend around the spring finger terminal ends and outboard the long edges thereof so as to provide additional skin engaging material, particularly between their inside long edges. A pair of slits 31 extend inward from where upper spring fingers diverge from lower spring fingers, forming a horizontal protrusion similar to flap (25) discussed hereinbefore. In order that the protrusion may separate or vertically protrude from the truss, as described hereinbefore (depicted as such in the drawing for illustrative purposes—it being understood that said protruding occurs when the truss is flexed across the bridge of the nose), slits 31 and valley 38′ preferably extend also through at least the cover layer of dilator 10.

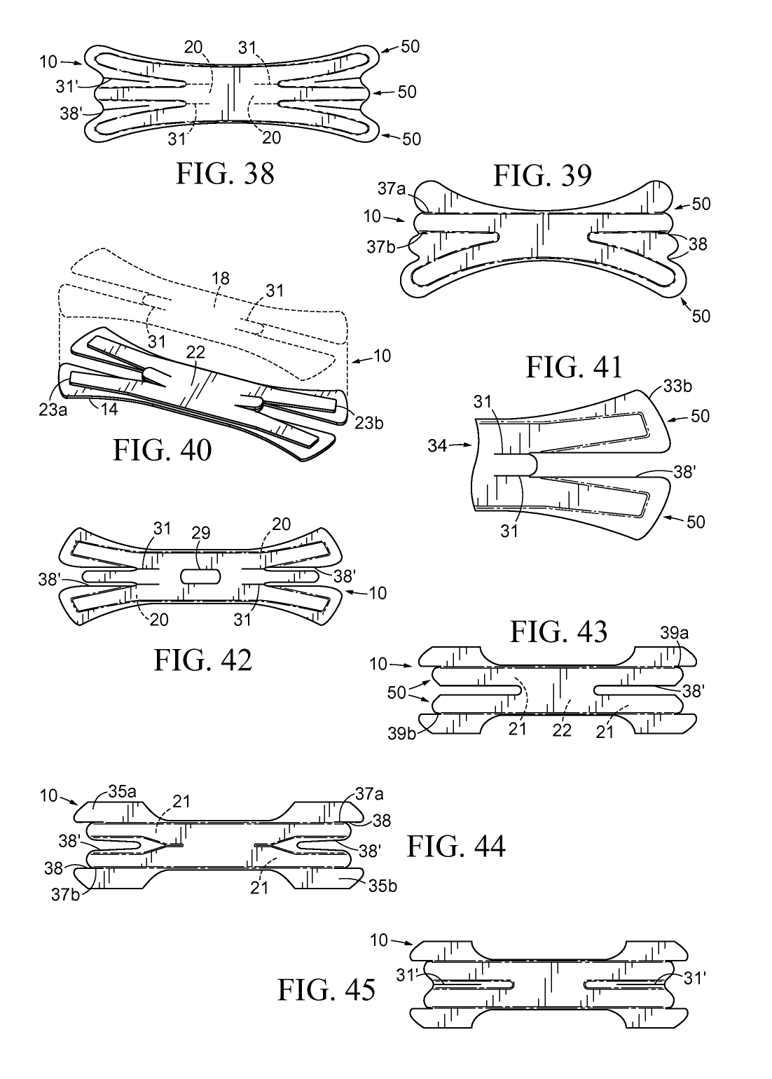

It will be apparent to the skilled artisan that each horizontal protrusion seen in FIGS. 40-41 may extend outward to form a middle spring finger interposed between upper and lower spring fingers, as seen in FIG. 42. Accordingly, slits 31 define the interior portion of each middle finger. Again, valleys, 38′ extend to about where upper fingers diverge from lower fingers, merging with and defining a portion of spring finger inside edges thereat, and the base and/or cover layers extend around the spring finger terminal ends and outboard the long edges. Material separations 29, 31, and 38′ are preferably formed concurrently, after assembly of the material laminate from which finished dilators 10 are die cut.

As seen in FIGS. 43-57, dilator 10 and resilient member 22 are generally rectangular in shape, similar to that illustrated previously in FIGS. 4, 16, and 18f. The drawing figures illustrate that spring finger terminal ends may be laterally closer together or farther apart, and the base and/or cover layers may include more or less material extending between upper and lower spring fingers so as to provide more or less skin engaging surface area. Depending upon the base and/or cover layer configuration, spring fingers may be made prior to assembling the vertical laminate of dilator 10, as seen, for example in FIGS. 44-48, 51-52, and 56-57, or after assembly of the vertical laminate, as seen, for example, in FIGS. 43, 49-50, and 53-55.

FIGS. 46, 49, 54 and 61 show tab extensions 35 and spring finger terminal ends extending to an imaginary line, as indicated by broken lines in the drawing figures, such that the truss end edges follow an inward angle between upper and lower corners thereof. The angle corresponds approximately to where the nose meets the cheek, as described hereinbefore.

FIGS. 51 and 57 illustrate that exterior material separations in the form of slits and/or valleys extending inward from an end edge of the truss to separate an end region may be omitted, and that the base and/or cover layers of dilator 10 may extend between adjacent spring fingers so as to have more skin engaging material thereat. However, the truss may include other material separations such as valleys 38, as shown, back cuts (37) or notches (39), not shown, so as to further define a horizontal protrusion at each end of opposing spring fingers, the protrusion capable of separating or protruding vertically, in part, from the truss when the dilator is flexed across the nose as described hereinbefore.

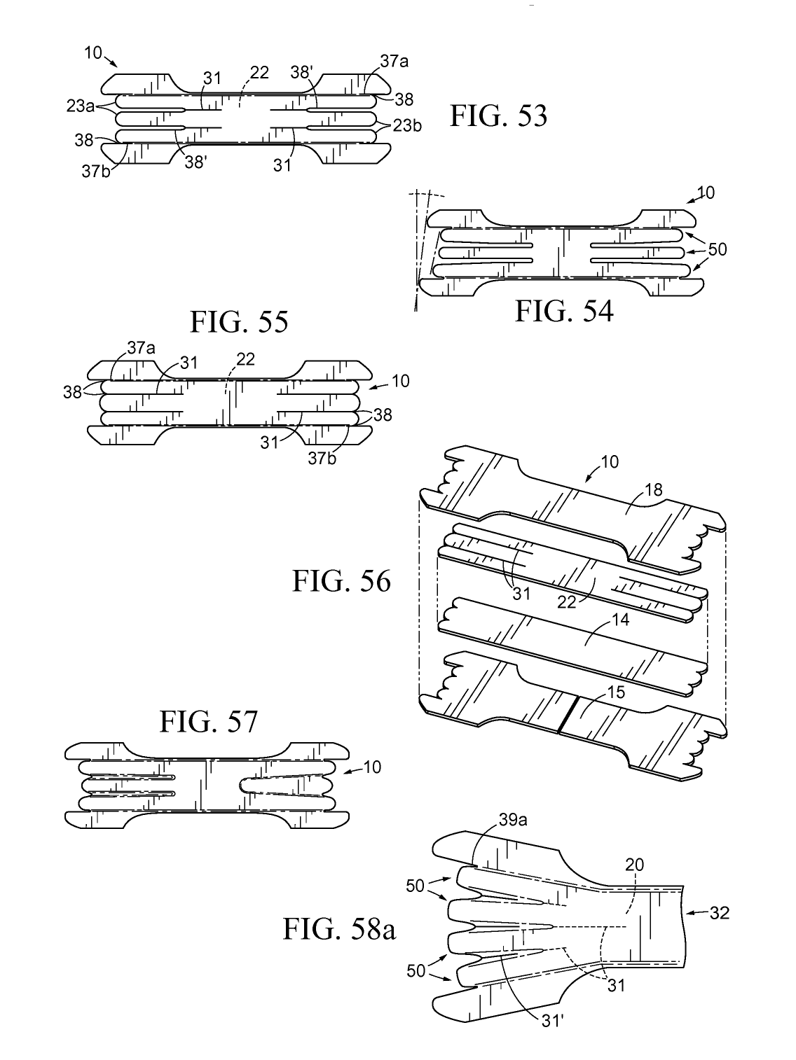

FIGS. 53-55 illustrate that valley 38′ and/or slits 31 may trifurcate the end regions of the truss so as to form three substantially parallel spring fingers. Alternatively, FIG. 56 illustrates that slits 31 may trifurcate the resilient member alone. FIG. 56 further illustrates that base member 14 may have the same peripheral shape as resilient member 22, as described hereinbefore.

As seen in FIG. 57, one end portion of resilient member 22 may be bifurcated and the opposing end portion may be trifurcated to form an asymmetric resilient member having first and second pluralities of spring fingers, respectively, extending into opposing end regions of the truss from a common center. The base and/or cover layer of dilator 10 extends between spring finger long edges and otherwise defines a substantially symmetric overall periphery, similar to that discussed previously with regard to FIG. 37.

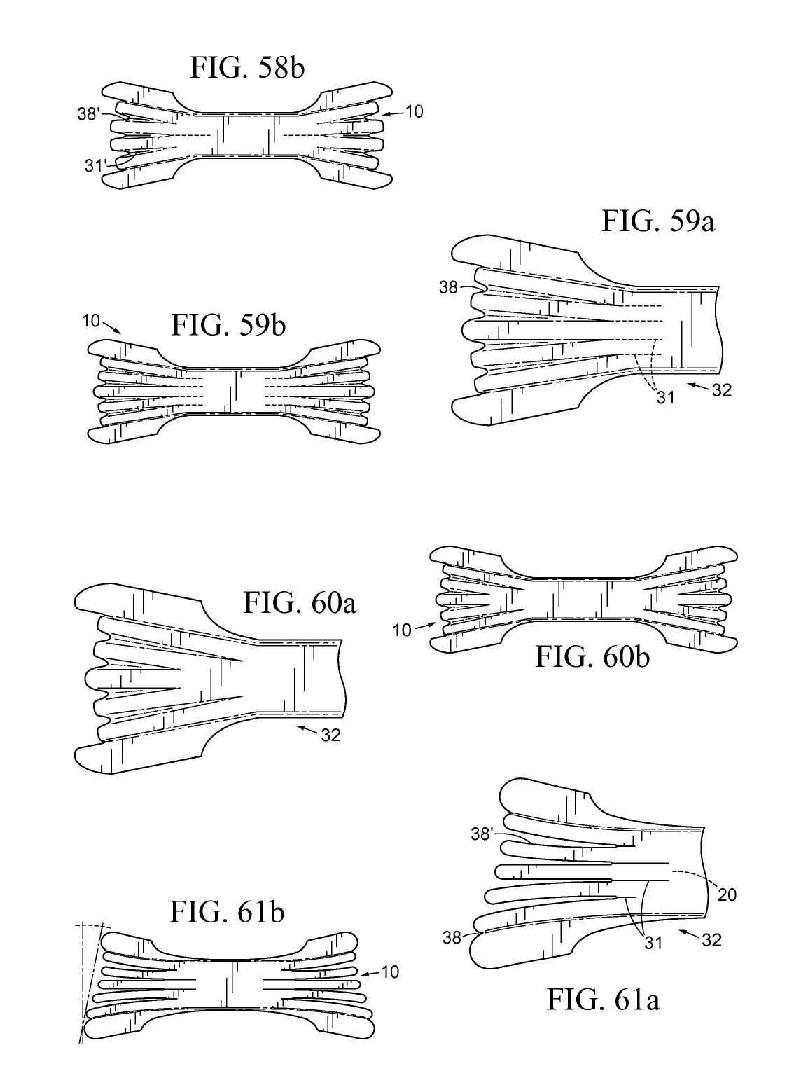

It will be apparent to the skilled practitioner that within the limitations of space, dilator spring biasing requirements, fabrication methods and suitable materials, any number of spring fingers may extend from a common center to discrete engagement contact points 50 in either end region, as seen, for example, in FIGS. 58-61. Similarly, as noted hereinbefore, any number of substantially rectangular parallel resilient members may comprise the resilient layer, subject to the same limitations.

FIGS. 58-61 illustrate a plurality of resilient member spring fingers extending from a common center into truss end regions 32 and 34. FIG. 58 shows that slits 31′ may extend into the base and/or cover layers of dilator 10, interposed between spring finger long edges. FIGS. 59 and 60 show that exterior material separations extending inward from an end edge of the truss to separate an end region may be omitted, the base and/or cover layers of dilator 10 extending substantially between adjacent spring fingers. However, as seen in % FIG. 61, material separations valleys 38′ form all spring finger long edges except those corresponding to the upper and lower long edges of resilient member 22. FIGS. 58, 60 and 61 illustrate that at least two spring fingers may branch out from a common spring finger. Alternatively, a spring finger may be seen as having a material separation extending inward from its terminal end, the material separation dividing that portion of the finger into two spring fingers.

In any case, the embodiments illustrate that spring finger components preferably radiate outward from the resilient member common center in a substantially uniform spread. That spread may vary in relation to the longitudinal centerline of the truss: centered to it or skewed to one side or the other. The spring fingers may have constant or gradient widths, may curve, etc., but together with any material separations are preferably configured such that any wider portion is positioned inboard of any narrower portion.

FIG. 61 shows tab extensions 35 and spring finger terminal ends extending to an imaginary line such that the truss end edges correspond to an inward angle between upper and lower corners of the long edges of dilator 10, the line or angle corresponding approximately to where the nose meets the cheek, as described hereinbefore.

The foregoing descriptions and illustrations are intended to reveal the scope and spirit of the present invention and should not be interpreted as limiting, but rather as illustrative of the inventive concepts thereof.

I claim:

1. A nasal dilator comprising a truss including:
a) first and second end regions adapted to engage outer wall tissues of first and second nasal passages, respectively, and an intermediate region interconnecting the first and second end regions;
b) a resilient member extending along the truss and secured to at least a portion of the first and second end regions, the resilient member comprising at least two spring fingers diverging and extending outward from a common center into the first end region, at least one spring finger diverging and extending outward from the common center into the second end region, at least one spring finger terminating at a portion of an outer lateral end edge of the truss at each of the first and second end regions, each terminating spring finger having a terminal end that conforms to said portion of the outer lateral end edge, the resilient member fabricated from a preferred material being substantially rigid in a direction parallel to a surface plane thereof; and
c) at least one of a non-resilient base layer or cover layer secured to one side of the resilient member for removably engaging the dilator to the outer wall tissues, said at least one of a base layer or cover layer defining at least a width of the truss extending outboard at least a portion of upper and lower long edges of the resilient member.

2. The nasal dilator of claim 1 and further including a slit extending vertically through the resilient member inward from a point located substantially where inside long edges of two laterally adjacent spring fingers extend from the common center, the slit formed in the resilient member prior to assembly of a vertical laminate from which the dilator is die cut.

3. The nasal dilator of claim 1 and further including a slit extending vertically through the resilient member and said at least one of a non-resilient base layer or cover layer inward from a point located substantially where inside long edges of two laterally adjacent spring fingers extend from the common center.

4. The nasal dilator of claim 1 wherein the resilient member comprises from three to five spring fingers extending into each of the first and second end regions.

5. The nasal dilator of claim 4 wherein portions of resilient member upper and lower long edges form outside long edges of uppermost and lowermost spring fingers, the dilator further including:
a) at least two of an exterior material separation extending inward from a lateral end edge of the truss, the material separations comprising a valley or a slit extending through said at least one of a non-resilient base layer or cover layer and the resilient member, the material separations forming spring finger inside long edges of said uppermost and lowermost spring fingers, the material separations further forming long edges of innermost spring fingers, said long edges being common to the resilient layer and the at least one of a non-resilient base layer or cover layer.

6. The nasal dilator of claim 1 wherein said at least one of a non-resilient base layer or cover layer extends in between at least a portion of laterally adjacent spring finger long edges.

7. The nasal dilator of claim 1 and further including at least one of an exterior material separation extending inward from a lateral end edge of the truss, the exterior material separation selected from the group consisting of:
a) a valley or a slit extending through said at least one of a non-resilient base layer or cover layer, the valley or slit interposed between two laterally adjacent spring fingers;
b) a valley formed at an intersection adjacent above or below an upper or lower corner of the spring finger terminal end;
c) a notch positioned parallel and adjacent an upper or lower long edge of the resilient member near a spring finger terminal end; or
d) a back cut extending vertically through at least the cover layer of the dilator inward from the lateral end edge, the back cut positioned at the intersection of a corresponding valley adjacent and parallel to an upper or lower long edge of the spring finger terminal end.

8. The nasal dilator of claim 7 wherein a long edge of the valley or slit interposed between two laterally adjacent spring fingers further extends approximately to a point at which an inside edge of one of said two laterally adjacent spring fingers diverges from the common center, the long edge merging with and defining a portion of the inside edge thereat, and further including:
a) a pair of slits extending into the common center from approximately where inside edges of two adjacent spring fingers diverge from the common center, the slits forming a flap or defining a portion of a middle spring finger thereat.

9. The nasal dilator of claim 1 wherein the resilient member is asymmetric so as to provide disparate dilating forces to opposing nasal outer wall tissues.

10. The nasal dilator of claim 1 wherein at least one spring finger terminates short of said outer lateral end edge.

11. The nasal dilator of claim 1 wherein at least one spring finger has at least one material separation extending inward from its terminal end, the material separation dividing said portion into two spring fingers extending from said at least one spring finger.

12. A nasal dilator comprising a laminate of vertical layers including at least one of a base layer or cover layer laminated to a resilient layer, the at least one of a base layer or cover layer substantially defining a periphery of the dilator, the resilient layer comprising at least three adjacent, spaced apart, substantially parallel resilient members.

13. The nasal dilator of claim 12 wherein at least two of the resilient members extend fully to first and second lateral end edges, respectively, of the dilator.

14. The nasal dilator of claim 12 wherein the resilient layer comprises three resilient members having substantially equal length, width, and thickness.

15. The nasal dilator of claim 12 wherein the resilient members have a dimensional relationship between width, thickness, and length, said dimensional relationship selected from the group consisting of:
a) a length of at least one resilient member is less than a length of at least one other resilient member;
b) a width of at least one resilient member is less than a width of at least one other resilient member;
c) a thickness of at least one resilient member is less than a thickness of at least one other resilient member.

16. The nasal dilator of claim 12 wherein the resilient members are spaced apart by a distance equal to or no greater than a width of a widest resilient member.

17. The nasal dilator of claim 12 wherein the resilient members have progressively shorter length.

18. An external nasal dilator comprising a truss, said truss comprising:
a) first and second end regions adapted to engage outer wall tissues of first and second nasal passages, respectively, and an intermediate region interconnecting the first and second end regions;
b) at least one of a flexible base layer or cover layer secured to one side of a resilient layer, the at least one of a flexible base layer or cover layer defining the first and second end regions and the intermediate region; and
d) a resilient layer comprising multiple parallel resilient members including a first outer resilient member secured along the length of the truss, a second outer resilient member spaced apart from and substantially parallel to the first outer resilient member and secured along the length of the truss, at least one intermediate resilient member secured along the length of the truss interposed between the first and second outer resilient members, the at least one intermediate resilient member being substantially parallel to the first and second outer resilient members.

19. The nasal dilator of claim 18 wherein:
a) the first outer resilient member and the second outer resilient member are equal to or no more than 20% different in length; and
b) the at least one intermediate resilient member is from 20% to 35% shorter than the first and second outer resilient members.

20. The nasal dilator of claim 18 wherein:
a) the first outer resilient member and the at least one intermediate resilient member are the same length; and
b) the second outer resilient member is 20-35% shorter than the first outer resilient member and the at least one intermediate resilient member.

21. The nasal dilator of claim 18 wherein the resilient member is placed on a first flat surface side of the truss and an adhesive material is disposed on a second flat surface side of the truss.

22. The nasal dilator of claim 18 wherein the at least one intermediate resilient member secured along the length of the truss interposed between the first and second outer resilient members comprises from two to five resilient members.

23. The nasal dilator of claim 18 wherein:
a) the resilient layer comprises three resilient members of equal length;
b) the resilient layer is placed on a first side of the truss and an adhesive is disposed on a second side of the truss; and
c) the truss further comprises a strip of backing material such that the resilient layer is interposed between the strip of backing material and the at least one of a non-resilient base layer or cover layer.
